(19) United States
(12) Patent Application Publication
(10) Pub. No.: US 2004/0146965 A1
(43) Pub. Date: Jul. 29, 2004

(54) Petri dish

(75) Inventor: Scott V. Brayton, Ames, IA (US)

Correspondence Address: MCKEE, VOORHEES & SEASE, P.L.C., 801 GRAND AVENUE, SUITE 3200, DES MOINES, IA, 50309-2721, US

(73) Assignee: Hach Company, Loveland, CO

(21) Appl. No.: 10/353,880

(22) Filed: Jan. 29, 2003

(51) Int. Cl. C12Q 1/04; C12M 1/34

(52) U.S. Cl. 435/34; 435/287.1; 435/288.3

(57) ABSTRACT

A self-contained petri dish container for growth of microorganism cultures. The petri dish container has a lid for the container which fits sufficiently snug to seal the container from contaminants, container pad for growth media containment positioned within the container and the container pad is made of sterile glass microfiber material, as opposed to conventional cellulose pads.

### FIELD OF THE INVENTION

The field of the present invention is an improved petri dish.

### BACKGROUND OF THE INVENTION

Petri dishes are, of course, known to microbiologists and/or medical technicians. The petri dish is used for harboring microorganism specimens, nutrient broth containing dyes, etc., then observing and counting the growth of microorganisms and finally, characterizing the grown microorganisms.

Typically, a petri dish will include some sort of an inert container with a snugly fit lid to prevent contamination. Inside is a cellulosic filter material to fit within the petri dish container in order to hold nutrient broth for the microorganisms.

Such dishes are commercially available from a variety of sources such as Pall Corporation, Millipore and Fisher Scientific. All have in common that they use pre-sterilized cellulose material filter pads. These pads are mostly sold prepackaged inside of the petri dish. Such pads are typically neutral in pH and are highly absorbent in order to absorb and contain a nutrient broth. While cellulosic fiber pads work fine for some uses, it has been discovered that for other uses, even the slight presence of materials remaining from the paper or cellulosic pad material production process, such as sulfites in minor amounts, will interfere with the growth of certain microorganism cultures such as coliforms.

Such interference, when it occurs, will provide a false test and can lead to an erroneous conclusion that there are no bacteria present when, in fact, the sulfites or other residual contaminants from the paper-making process simply inhibit microorganism growth. This problem has been found especially pertinent to petri dish tests for detecting and distinguishing E-coli and total coliforms such as those tests described in Grant, U.S. Pat. No. 5,849,515. In the test system of the Grant '515 patent, there is a selective culture medium which permits simultaneous detection of total coliform and Escherichia coli in a test sample with a single growth phase incubation period. The culture medium includes the required components of: (i) carbon nutrients, (ii) a nitrogen nutrient, (iii) a source of metabolizable potassium, (iv) a source of metabolizable phosphate, (v) vitamins, (vi) minerals, (vii) amino acids, (vii) sodium pyruvate, (ix) a bactericidal system selective for non-coliform bacteria which includes methylene blue, erythromycin and an azide, and (x) a sensible indicator selectively metabolized by Escherichia coli to the exclusion of other coliforms. The description of medium described in Grant, U.S. Pat. No. 5,849,515 is incorporated herein by reference.

The medium described in that patent is commercially sold by Hach Company, Loveland, Colo. as m-ColiBlue24.

It is a primary objective of the present invention to provide a petri dish/pad absorbent medium which is not only sterile but is free from residual contaminants normally associated with paper processing.

A further object of the present invention is to provide a petri dish/pad matrix combination which is of non-cellulosic origin.

A further objective of the present invention is to provide a pad medium material which is still capable of high absorbency of nutrient medium but which is also free of cellulosic paper processing contaminants, and which will allow detection of coliforms, and distinguishing detection of E-coli from other coliforms.

The method and means of accomplishing each of the above will become apparent from the detailed description of the invention which follows.

### BRIEF DESCRIPTION OF THE DRAWINGS

FIG. 1 is a perspective view of petri dish of the present invention, shown with the non-cellulose matrix pads and further shown with the lid separated from the container.

FIG. 2 shows a cross section of the petri dish microfiber pad matrix combination of FIG. 1.

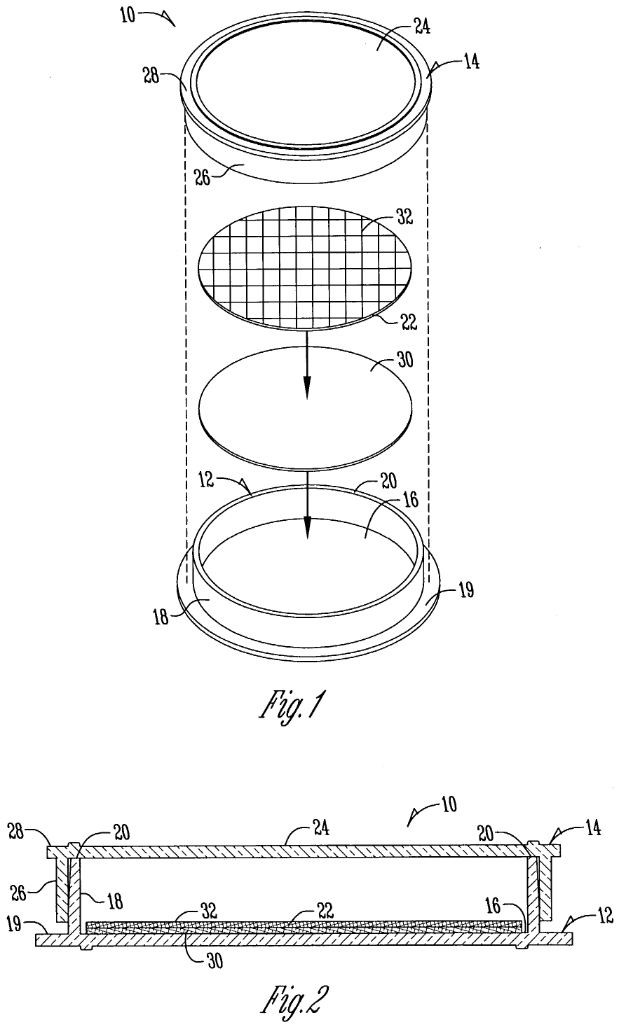

### SUMMARY OF THE INVENTION

A self-contained petri dish container for growth of microorganism cultures. The petri dish container has a lid for the container which fits sufficiently snug to seal the container from contaminants, a container pad for growth media containment positioned within the container, and the container pad is made of sterile glass microfiber material, as opposed to conventional cellulose pads.

### DETAILED DESCRIPTION OF A PREFERRED EMBODIMENT

Referring now to the drawings, the petri dish 10 consists of a container 12 and a lid 14 for the container, each of these parts being of an inert organic polymeric resin and of unitary molded construction. At least the lid 14 should be transparent to enable inspection of the culture grown in the petri dish. It is preferred that both the lid and the container be molded of a transparent inert polymeric resin such as transparent polystyrene.

The container has a flat, round bottom wall 16 from which there extends perpendicularly upwardly a substantially cylindrical side wall 18. In one embodiment, extending radially outwardly from and substantially coplanar with the bottom wall 16 is a circular flange 19. The outer surface of the side wall is tapered inwardly at its upper extremity, as shown at 20, whereby this upper portion of the outer surface of side wall is of frustoconical shape. Such a suitable petri dish is described in U.S. Pat. No. 4,160,700.

The lid 14 of the petri dish has a flat, round top wall 24 which has extending perpendicularly downwardly therefrom a substantially cylindrical side wall 26 having a depth less than the height of side wall 18 of the container 10. Extending radially outwardly and substantially coplanar with the top wall 24 is a circular flange 28 having an outer diameter substantially less than the outer diameter of the flange 19 of the container.

Located within container 10 such that it rests on flat, round bottom wall 16 is a container pad 30 which will absorb growth media. Container pad 30 is not a cellulosic pad material as conventionally used, but instead is a glass microfiber material. Glass microfiber filters are generally known, but have not heretofore been used in petri dishes. Such glass microfiber filters are resistant to weakening or disruption of the fibrous matrix by either inorganic or organic liquids. Typically they are multi-porous and borosilicate glass microfibers. In the highest, most advanced filter types, they are available in sterile form and are binder free, are virtually non-aging and are inert. Suitable materials can be obtained from Whatman. One that is particularly suitable is a Whatman filter GF/D 2.7 μm. Such products, as far as the applicant knows, have not been used before for this purpose and are described by Whatman as high purity borosilicate glass microfiber filters and/or protection for membranes. For further information on Whatman glass fiber materials, see http:www.Whatman.com.

Applicant's invention of the combination of microfiber glass material and petri dishes as a replacement for conventional cellulosic fiber material and petri dishes was arrived at to solve a problem existing with Hach Company M-ColiBlue24, a medium used in detecting total coliforms and in distinguishing E-coli from other coliforms. In this system, one uses a selective culture medium which permits simultaneous detection of total coliform and Escherichia coli in a test sample with a single growth phase incubation period. The culture medium includes the required components of: (i) carbon nutrients, (ii) a nitrogen nutrient, (iii) a source of metabolizable potassium, (iv) a source of metabolizable phosphate, (v) vitamins, (vi) minerals, (vii) amino acids, (viii) sodium pyruvate, (ix) a bactericidal system selective for non-coliform bacteria, and (x) a suitable indicator selectively metabolized by Escherichia coli to the exclusion of other coliforms; and the optional components of: (xi) a source of metabolizable magnesium, (xii) sodium chloride as a recovery agent, (xii) a nonionic surfactant, (xiii) a contrast promoter effective for enhancing the color differentiation between Escherichia coli colonies growing in the medium and other bacterial colonies growing in the medium induced by the suitable indicator, and (xiv) a highlighting dye for enhancing the visual differentiation of coliform colonies relative to the culture medium.

False negatives in the test give rise to complaints that m-ColiBlue24 did not work. Careful investigation by researchers at Hach revealed that the problem was not either the method or the detection medium, but rather the residual contaminants from cellulosic filters conventionally used. When those were replaced with multi-porous microfiber borosilicate material preferred in the present invention, the problems ended, the medium worked satisfactorily and detection not only was successful but was enhanced.

In actual operation after the nutrient media is placed on pad 30, the material containing the sample for detection is filtered through the sterilized membrane with grid, 32, and the sterilized membrane 22 with the grid 32 is placed over the pad. The sterilized membrane 32 has a grid on it to facilitate colony counting.

### EXAMPLE

In this experiment, several brands of membrane filters were tested with a given E-coli culture dilution. These filters were placed on either broth-saturated Pall-Gelman pad or a special microfiber borosilicate glass pad, a Whatman GF/D glass fiber filter disc. The results were dramatic; healthy growth was observed on the glass pad and no growth on the cellulose pad. This was repeated with chlorine-stressed E-coli and the same results were observed. Then cultures from the standard m-CB QC procedure were used. These included Enterobacter and Klebsiella. In each case, the growth of organisms was stronger and resolution of red and blue colonies was more distinct on the glass fiber pad than previously observed. Colony counts could be performed at 17 hours in cases where it required 24 hours on the conventional pad. This illustrates another advantage of the invention.

These tests revealed the reason why the cellulose pad was performing poorly. It was due to two factors. The cellulose pad was only absorbing about 1.4 ml of broth instead of the recommended 2 ml. Thus the remaining broth was not available for colony growth when the dish was inverted for incubation. The presence of reducing agents known to be inhibitory to colony growth was qualitatively determined in the cellulosic pads. The glass pad absorbs essentially all the 2 ml of broth which is added and the material is chemically inert.

The unique combination of conventional petri dishes and the glass microfiber multi-porous borosilicate pad when used with Hach m-ColiBlue24, provided superior results in both coliform detection and E-coli detection. It, therefore, can be seen that the invention accomplishes at least all of its stated objectives.

What is claimed is:

1. In a self-contained petri dish container for growth of microorganism cultures, said petri dish container having a lid for the container, and within the container a container pad for growth media containment, the improvement comprising:
a container pad of glass microfiber material.

2. The self-contained petri dish of claim 1 wherein the glass microfiber material is multi-porous.

3. The self-contained petri dish of claim 1 wherein the multi-porous microfiber material is borosilicate material.

4. The self-contained petri dish of claim 3 wherein the multi-porous borosilicate material is binder free.

5. The self-contained petri dish of claim 1 which also contains a sterile membrane filter pad.

6. The self-contained petri dish of claim 5 wherein the sterile membrane filter pad is grid marked.

7. A self-contained petri dish container for growth of microorganism cultures, comprising:
an inert container;
an inert lid for the container;
a container pad for growth media containment positioned within the container; and
said container pad being glass microfiber material.

8. The self-contained petri dish of claim 7 wherein the glass microfiber material is multi-porous.

9. The self-contained petri dish of claim 8 wherein the multi-porous microfiber material is borosilicate material.

10. The self-contained petri dish of claim 9 wherein the multi-porous borosilicate material is binder free.

11. The self-contained petri dish of claim 7 which also contains a sterile membrane filter pad.

12. The self-contained petri dish of claim 11 wherein the sterile membrane filter pad is grid marked.

13. A process of growth of coliforms and detecting E-coli as distinct from other coliforms comprising:
selecting a petri dish container inert to microorganism cultures, said petri dish having a lid and a container pad for growth media containment, said container pad being of glass microfiber material;
applying a culture medium to said container pad which provides for selective growth of coliforms and simultaneous detection of coliforms and E-coli as distinct from other coliforms;
placing a membrane filter over the container pad containing said culture medium; and
placing a sample containing bacteria for detection on said membrane filter pad.

14. The process of claim 13 wherein the glass microfiber material is multi-porous.

15. The process of claim 14 where in the glass microfiber material is borosilicate material.

16. The process of claim 15 wherein the glass microfiber material is binder free.
